(19) United States
(12) Patent Application Publication
(10) Pub. No.: US 2016/0084850 A1
(43) Pub. Date: Mar. 24, 2016

(54) METHOD FOR QUANTIFYING GLYCATED HEMOGLOBIN

(71) Applicant: PANASONIC HEALTHCARE HOLDINGS CO., LTD., Tokyo (JP)

(72) Inventors: Takashi ENDOH, Ehime (JP); Nobuyuki SHIGETOH, Ehime (JP)

(21) Appl. No.: 14/890,375

(22) PCT Filed: Feb. 27, 2014
(86) PCT No.: PCT/JP2014/001063

§ 371 Date: Nov. 10, 2015

(30) Foreign Application Priority Data
May 31, 2013 (JP) 2013-114986

(51) Int. Cl. G01N 33/72

(52) U.S. Cl. CPC G01N 33/723; G01N 2333/805; G01N 2440/38; G01N 2333/95; G01N 2333/908; G01N 2333/954

(57) ABSTRACT

A method for quantifying glycated hemoglobin (HbA1c) contained in a sample, the method including: (a) a process for mixing the sample with a protease in the presence of a composition of a cationic surfactant and a tetrazolium salt to obtain an aqueous glycated peptide solution containing a glycated peptide; (b) a process for mixing the aqueous glycated peptide solution obtained in process (a) with a fructosyl peptide oxidase to obtain hydrogen peroxide, the aqueous glycated peptide solution here containing the composition; and (c) a process for calculating the concentration of the glycated hemoglobin (HbA1c) on the basis of the amount of hydrogen peroxide solution obtained in process (b).

### TECHNICAL FIELD

The present invention relates to a method for quantifying glycated hemoglobin contained in a sample.

### BACKGROUND ART

Glycated hemoglobin (hereinafter, referred to as “HbA1c”) is a kind of glycated protein formed by nonenzymatically binding a glucose to a hemoglobin A (hereinafter, referred to as “HbA”) included in a red blood cell. The HbA1c is used as a diabetes marker.

Patent document 1 discloses a method for quantifying HbA1c by an enzyme assay method. More particularly, the method disclosed in patent document 1 comprises the following steps (a) and (b).

(a) decomposing HbA1c with protease to produce fructosyl peptide; and

(b) quantifying the fructosyl peptide produced in the step (a) with fructosyl peptide oxidase (hereinafter, referred to as “FPOX”).

Patent document 2 also discloses a method for obtaining fructosyl peptide. This method is characterized by that a sample containing protein such as HbA1c is subjected to a protease treatment in the presence of a tetrazolium compound. Patent document 2 further discloses that not only the tetrazolium compound but also a surfactant promotes the protease treatment more.

Patent document 3 also discloses a method for obtaining fructosyl peptide. In this method, HbA1c is subjected to a protease treatment in the presence of an isothiazoline derivative and a surfactant.

### PRIOR ART DOCUMENTS

### Patent Documents

Patent Document 1: U.S. Patent Application Publication No. 2007/0037243

Patent Document 2: U.S. Patent Application Publication No. 2003/0186346

Patent Document 3: Canadian Patent Application Publication No. 2 806 261 A

Patent Document 4: U.S. Pat. No. 7,235,378

Patent Document 5: U.S. Pat. No. 7,855,079

### SUMMARY OF THE INVENTION

### Problems to be Solved by the Invention

In the enzyme assay method, the step (a) is required to be performed rapidly. In greater detail, it is necessary to more increase the decomposition rate of HbA1c, which is caused by a protease.

Furthermore, the chemical reagent used in the step (a) must not harm the step (b). In more detail, the chemical reagent used in the step (a) must not inactivate the FPOX.

The purpose of the present invention is to provide a method for quantifying glycated hemoglobin rapidly.

The present invention relates to a method for quantifying glycated hemoglobin (HbA1c) contained in a sample, the method comprising the steps of:

(a) mixing the sample with a protease in the presence of a composition of a cationic surfactant and a tetrazolium salt to obtain a glycated-peptide aqueous solution containing a glycated peptide;

(b) mixing the glycated-peptide aqueous solution obtained in the step (a) with FPOX (fructosyl peptide oxidase) to obtain hydrogen peroxide; wherein the glycated-peptide aqueous solution contains the composition; and

(c) calculating a concentration of the glycated hemoglobin (HbA1c) on the basis of an amount of the hydrogen peroxide obtained in the step (b).

The present invention relates to a method for quantifying glycated hemoglobin (HbA1c) contained in a sample, the method comprising steps of:

(a) mixing the sample with a protease and FPOX (fructosyl peptide oxidase) in the presence of a composition of a cationic surfactant and a tetrazolium salt to obtain hydrogen peroxide; and

(b) calculating a concentration of the glycated hemoglobin (HbA1c) on the basis of an amount of the hydrogen peroxide obtained in the step (a).

The sprits of the present invention include a composition for activating a protease containing a cationic surfactant a tetrazolium salt.

### EFFECTS OF THE INVENTION

The present invention provides a method for quantifying glycated hemoglobin rapidly.

### BRIEF DESCRIPTION OF THE DRAWINGS

FIG. 1A shows the results of the test example 1.

FIG. 1B shows the results of the test example 1.

FIG. 2A shows the results of the test example 2.

FIG. 2B shows the results of the test example 2.

FIG. 3 shows the results of the test example 3.

FIG. 4 shows the results of the test example 4.

FIG. 5 shows the results of the test example 5.

### DETAILED DESCRIPTION OF THE INVENTION

The embodiments of the present invention are described below.

(Embodiment 1)

First, the embodiment 1 is described.

(Step (a))

In the step (a), a sample containing HbA1c is mixed with a protease in the presence of a composition of a cationic surfactant and a tetrazolium salt. Preferably, the sample is an aqueous solution.

The HbA1c is decomposed by the protease to produce a glycated peptide. An example of the glycated peptide is a fructosyl peptide. An example of the fructosyl peptide is fructosyl-valine-histidine (hereinafter, referred to as “Fru-Val-His”). In this way, obtained is an aqueous solution containing a glycated peptide.

An example of the sample containing HbA1c is blood derived from a human. The dilution of the blood derived from a human is also included in the sample containing HbA1c.

An example of the cationic surfactant is a quaternary ammonium salt, an alkylamine salt or a pyridine derivative. A preferable cationic surfactant is a quaternary ammonium salt, which is represented by the chemical formula R₁R₂R₃R₄N⁺X⁻.

Preferably, R₁ is an alkyl group having a carbon number of not less than 8 and not more than 18. Preferably, R₂, R₃ and R₄ are lower alkyl groups independently. More preferably, R₂, R₃ and R₄ are a methyl group or an ethyl group independently. A methyl group is still more preferable. X represents halogens. Preferably, X represents chlorine or bromine.

Examples of a preferable cationic surfactant are listed below.

Trimethyl octyl ammonium chloride

Trimethyl decyl ammonium chloride

Trimethyl dodecil amonium chloride

Trimethyl tetradecyl ammonium chloride

Trimethyl cetyl ammonium chloride

Trimethyl stearyl ammonium chloride

Trimethyl octyl ammonium bromide

Trimethyl decyl ammonium bromide

Trimethyl dodecil amonium bromide

Trimethyl tetradecyl ammonium bromide

Trimethyl cetyl ammonium bromide

Trimethyl stearyl ammonium bromide

Examples of a preferable tetrazolium salt are listed below.

2-(4-Iodophenyl)-3-(4-nitrophenyl)-5-(2,4-disulfophenyl)-2H-tetrazolium, monosodium salt (WST1)

2-(4-Iodophenyl)-3-(2,4-dinitrophenyl)-5-(2,4-disulfophenyl)-2H-tetrazolium, monosodium salt (WST-3)

2-(2-Benzothiazolyl)-3-(4-carboxy-2-methoxyphenyl) -5[4-[(2-sodiosulfoethyncarbamoyl]phenyl]-2H-tetrazol-3-ium (WST-4)

2,2′-(3,3′-dimethoxy-4,4′-biphenylylene) bis[3-(2-benzothiazolyl) -5-[4-[N-[2-(sodiooxysulfonyl) ethyl]-N-(2-sulfoethyl) carbamoyl]phenyl]-2H-tetrazole -3-ium] (WST-5)

5-[2,4-Bis(sodiooxysulfonyl)phenyl]-3-(2-methoxy-4-nitrophenyl) -2-(4-nitrophenyl)-2H-tetrazole-3-ium (WST-8)

2-(4-Nitrophenyl)-5-phenyl-3[4-(4-sulfophenylazo)-2-sulfophenyl]-2H-tetrazolium, monosodium salt (WST-9)

2,5-Di(4-nitrophenyl)-3-[4-(4-sulfophenylazo)-2-sulfophenyl]-2H-tetrazolium, monosodium salt (WST-10)

2-(4-Nitrophenyl)-5-(2-sulfophenyl)-3-[4-(4-sulfophenylazo)-2-sulfophenyl]-2H-tetrazolium, disodium salt (WST-11)

An example of the protease is thermolysin, papain, chymotrypsin, subtilisin, caspase, pepsin, or cathepsin D. Thermolysin and papain are preferable.

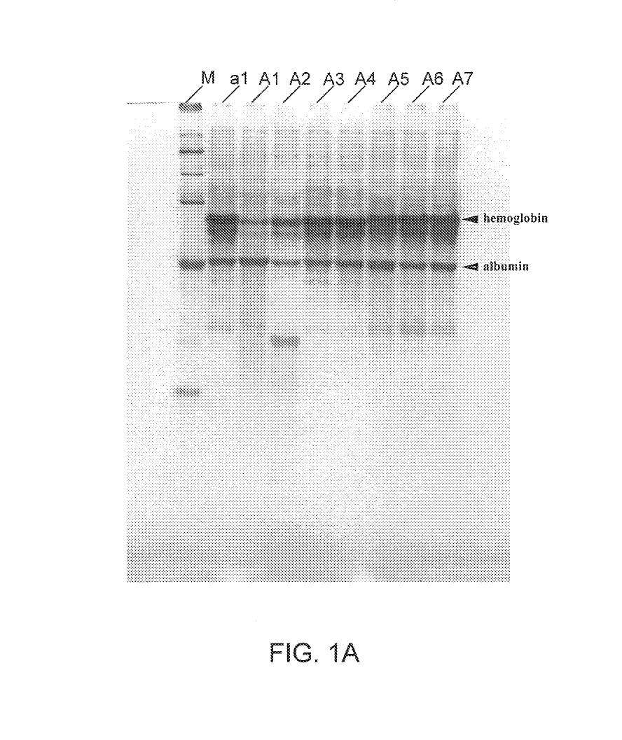

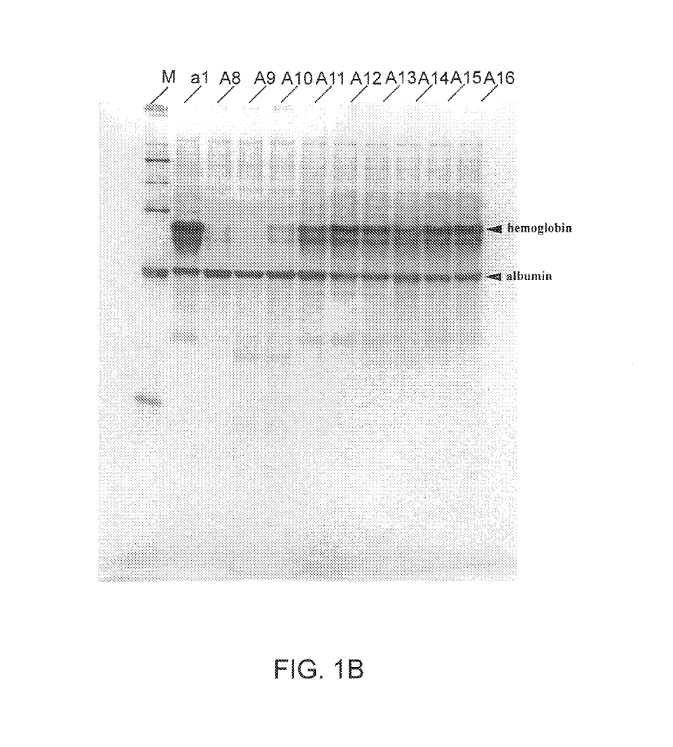

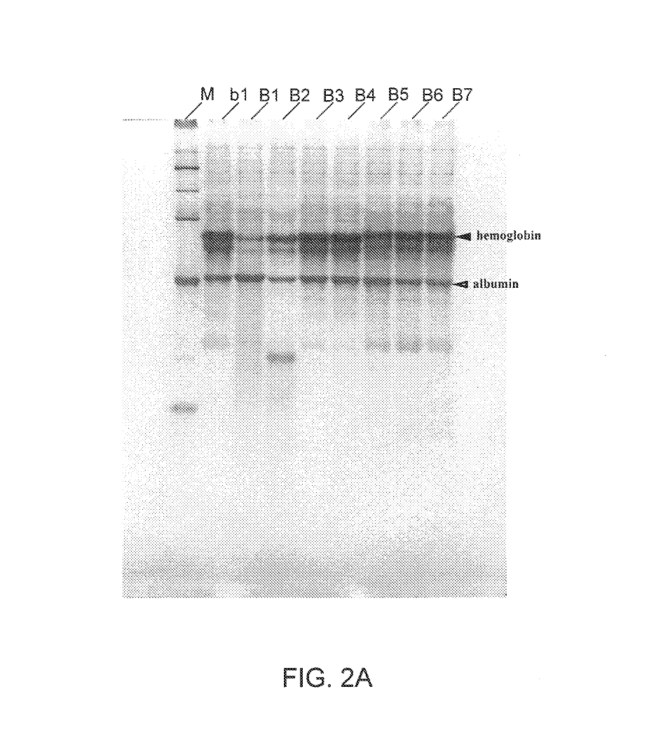

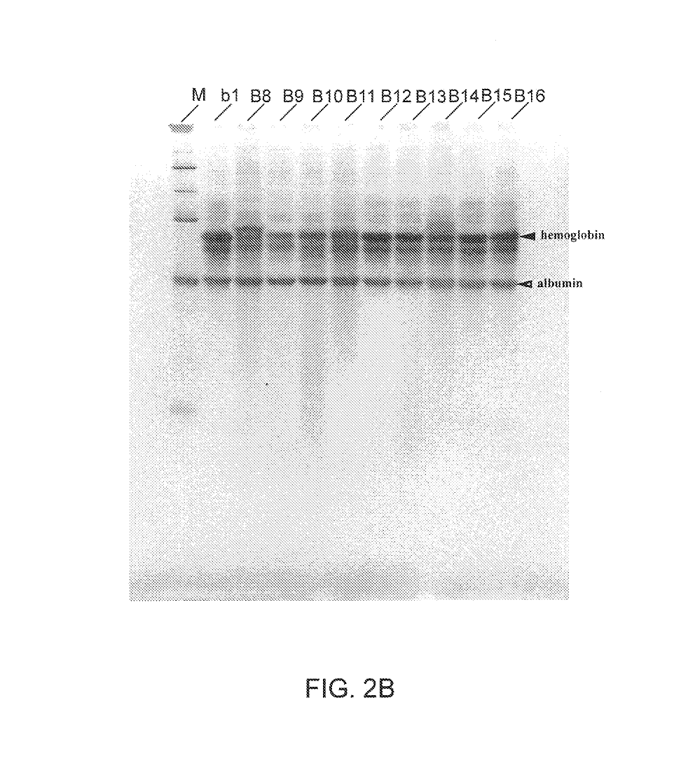

As demonstrated in the sample solutions A8-A10 and B8-B10 included in the test examples 1 and 2, which are described later, the combination of the cationic surfactant and the tetrazolium salt significantly improves a decomposition rate of HbA1c. In other words, the synergistic effect of the cationic surfactant and the tetrazolium salt significantly improves the decomposition rate of HbA1c. See FIG. 1A, FIG. 1B, FIG. 2A and FIG. 2B.

As demonstrated in the sample solutions A1 and B1 included respectively in the test examples 1 and 2, an anionic surfactant such as sodium dodecyl sulfate (hereinafter, referred to as “SDS”) also improves the decomposition rate of HbA1c. However, an anionic surfactant must not be used in the present invention. The reason is described in the description of the step (b).

As demonstrated in the sample solutions A2 and B2 included respectively in the test examples 1 and 2, the decomposition rate of HbA1c is not improved when the cationic surfactant is used alone. Similarly, as demonstrated in the sample solutions A5-A7 and B5-B7 included in the test examples 1 and 2, the decomposition rate of HbA1c is not improved when the tetrazolium salt is used alone.

As demonstrated in the sample solutions A3, A4, B3 and B4 included in the test examples 1 and 2, the decomposition rate of HbA1c is not improved when a nonionic surfactant is used instead of the cationic surfactant. As demonstrated in the sample solutions A11-A16 and B11-B16 included in the test examples 1 and 2, the decomposition rate of HbA1c is not improved, even when a nonionic surfactant is used together with the tetrazolium salt,.

(Step (b))

The step (b) is performed after the step (a).

The glycated peptide aqueous solution obtained in the step (a) is mixed with fluctosylpeptide oxidase (hereinafter, referred to as “FPOX”). As disclosed in patent document 2, the glycated peptide reacts with the FPOX to produce hydrogen peroxide. In the step (b), the glycated peptide aqueous solution contains the composition of the cationic surfactant and the tetrazolium salt, similarly to the step (a).

As demonstrated in the sample solutions C8-C10, D8-D10 and E8-E10 included in the test examples 3-5, which are described later, the glycated peptide reacts with the FPOX more rapidly in a case where the composition of a cationic surfactant and the tetrazolium salt is used, compared to the case where the cationic surfactant is used alone (See sample solutions C2, D2, and E2).

An anionic surfactant improves the decomposition rate of HbA1c. However, the anionic surfactants inactivate the FPOX, as demonstrated in the sample solutions C1, D1 and E1, which is included in the test example 3, 4 and 5, which are described later. For this reason, in case where the anionic surfactant is used, then the HbA1c failed to be quantified.

(Step (c))

The step (c) is performed after the step (b).

The amount of the hydrogen peroxide produced in the step (b) is proportional to the amount of the HbA1c contained in the sample, as disclosed in patent document 4 (see column 8 lines 6-37) and patent document 5 (see column 10 line 63-column 11 line 7). These patent documents are incorporated herein by reference. The following home page discloses that the amount of the hydrogen peroxide produced in the step (b) is proportional to the amount of the HbA1c contained in the sample.

http ://www.sekisuimedical.jp /english/business/diagnostics/biochemistry/hb a1c/index.html

Accordingly, the amount of the HbA1c contained in the sample is calculated on the basis of the amount of the hydrogen peroxide with use of a standard curve obtained in advance. In this way, the HbA1c contained in the sample is quantified. In other words, the concentration of the HbA1c is measured.

(Embodiment 2)

Then, the embodiment 2 is described.

(Step (d))

In the step (d), the sample containing HbA1c is mixed with the protease and the FPOX in the presence of the composition of the cationic surfactant and the tetrazolium salt to obtain hydrogen peroxide. Preferably, the sample is an aqueous solution.

(Step (e))

The step (e) is performed after the step (d). In the step (e), HbA1c is quantified on the basis of the quantity of the hydrogen peroxide obtained in the step (d)

In the step (d), the step (a) and the step (b) are performed simultaneously. The step (e) is identical to the step (c). Accordingly, the detailed descriptions of the step (d) and the step (e) are omitted.

### TEST EXAMPLE

(Preparation of the sample solution)

Whole human blood (available from BIOPREDIC INTERNATIONAL) was diluted to make a ten-fold dilution thereof. In this way, a sample solution containing hemoglobin was prepared. Hereinafter, this sample solution is referred to as “Sample Solution P”.

(Test example 1)

(Preparation of the sample solution)

The following sample solutions A1-A16 were prepared. A comparative sample solution al was also prepared. The source of the used reagents is listed below.

Thermolysin: Wako Pure Chemical Industries, Ltd. SDS: Wako Pure Chemical Industries, Ltd. TTAB (tetradecyl trimethyl ammonium bromide): Wako Pure Chemical Industries, Ltd. TritonX-100: Wako Pure Chemical Industries, Ltd. Tween20: Wako Pure Chemical Industries, Ltd. WST-3: Dojindo Laboratories WST-4: Dojindo Laboratories WST-5: Dojindo Laboratories

(Sample Solution A1) Sample solution P Thermolysin PBS solution: 150,000 U/mL SDS PBS solution: 10% by weight

(Sample Solution A2) Sample solution P Thermolysin PBS solution: 150,000 U/mL TTAB: 10% by weight

(Sample Solution A3) Sample solution P Thermolysin PBS solution: 150,000 U/mL TritonX-100 PBS solution: 10% by weight

(Sample Solution A4) Sample solution P Thermolysin PBS solution: 150,000 U/mL Tween20 PBS solution: 10% by weight

(Sample Solution A5) Sample solution P Thermolysin PBS solution: 150,000 U/mL WST-3 water solution: 2% by weight

(Sample Solution A6) Sample solution P Thermolysin PBS solution: 150,000 U/mL WST-4 PBS solution: 0.5% by weight

(Sample Solution A7) Sample solution P Thermolysin PBS solution: 150,000 U/mL WST-5 PBS solution: 2% by weight

(Sample Solution A8) Sample solution P Thermolysin PBS solution: 150,000 U/mL TTAB 10%: by weight WST-3 PBS solution: 2% by weight

(Sample Solution A9) Sample solution P Thermolysin PBS solution: 150,000 U/mL TTAB 10%: by weight WST-4 PBS solution: 0.5% by weight

(Sample Solution A10) Sample solution P Thermolysin PBS solution: 150,000 U/mL TTAB: 10% by weight WST-5 PBS solution: 2% by weight

(Sample Solution A11) Sample solution P Thermolysin PBS solution: 150,000 U/mL TritonX-100 PBS solution: 10% by weight WST-3 PBS solution: 2% by weight

(Sample Solution A12) Sample solution P Thermolysin PBS solution: 150,000 U/mL TritonX-100 PBS solution: 10% by weight WST-4 PBS solution: 0.5% by weight

(Sample Solution A13) Sample solution P Thermolysin PBS solution: 150,000 U/mL TritonX-100 PBS solution: 10% by weight WST-5 PBS solution: 2% by weight

(Sample Solution A14) Sample solution P Thermolysin PBS solution: 150,000 U/mL Tween20 PBS solution: 10% by weight WST-3 PBS solution: 2% by weight

(Sample Solution A15) Sample solution P Thermolysin PBS solution: 150,000 U/mL Tween20 PBS solution: 10% by weight WST-4PBS solution 0.5%: by weight

(Sample Solution A16) Sample solution P Thermolysin PBS solution: 150,000 U/mL Tween20 PBS solution: 10% by weight WST-5 PBS solution: 2% by weight

(Comparative Sample Solution a1) Sample solution P Thermolysin PBS solution: 150,000 U/mL

(HbA1c Decompsition Reaction Using Thermolysin)

The temperature of the sample solutions A1-A16 and the comparative sample solution al was maintained at 37 degrees Celsius for ten minutes and the reaction occurred.

(Comparison of the Decomposition Reaction Progress)

After the reaction, the sample solutions A1-A16 and the comparative sample solution al were subjected to polyacrylamide gel electrophoresis (PAGE). FIG. 1A and FIG. 1B show the electrophoresis patterns. The character “M” depicted in FIG. 1A and FIG. 1B indicates a protein maker.

The degree of the HbA1c decomposition is reflected in the color strength of the band near 64.5 KDa, which corresponds to hemoglobin molecular region included in the electrophoresis pattern. If the color strength of the band near 64.5 KDa decreases, the more amounts of HbA1c is decomposed.

As is clear from the electrophoresis patterns shown in FIG. 1A and FIG. 1B, the more amount of HbA1c was decomposed in the sample solutions A1, A8, A9 and A10, compared to the comparative sample solution a1. The more amount of HbA1c was decomposed in the sample solutions A8, A9 and A10, compared to the sample solutions A2, A5, A6, and A7.

SDS (Sample solution A1), the composition of TTAB and WST-3 (Sample solution A8), the composition of TTAB and WST-4 (Sample solution A9) and the composition of TTAB and WST-5 (Sample solution A10) improve the decomposition rate of HbA1c using thermolysin.

The composition of TTAB and WST-3 (Sample solution A8) improves the decomposition rate of HbA1c using thermolysin, compared to the case where TTAB was used alone (Sample solution A2) and the case where WST-3 was used alone (Sample solution A5).

The composition of TTAB and WST-4 (Sample solution A9) improves the decomposition rate of HbA1c using thermolysin, compared to the case where TTAB was used alone (Sample solution A2) and the case where WST-4 was used alone (Sample solution A6).

The composition of TTAB and WST-5 (Sample solution A10) improves the decomposition rate of HbA1c using thermolysin, compared to the case where TTAB was used alone (Sample solution A2) and the case where WST-5 was used alone (Sample solution A7).

(Test Example 2)

(Preparation of the Sample Solution)

The following sample solutions B1-B16 were prepared. A comparative sample solution b1 was also prepared. The source of the used reagents is listed below.

Papain: Roche Diagnostics K.K. SDS: Wako Pure Chemical Industries, Ltd. TTAB (tetradecyl trimethyl ammonium bromide): Wako Pure Chemical Industries, Ltd. TritonX-100: Wako Pure Chemical Industries, Ltd. Tween20: Wako Pure Chemical Industries, Ltd. WST-3: Dojindo Laboratories WST-4: Dojindo Laboratories WST-5: Dojindo Laboratories

(Sample Solution B1) Sample solution P Papain PBS solution: 300 U/mL SDS PBS solution: 10% by weight

(Sample Solution B2) Sample solution P Papain PBS solution: 300 U/mL TTAB: 10% by weight

(Sample Solution B3) Sample solution P Papain PBS solution: 300 U/mL TritonX-100 PBS solution: 10% by weight

(Sample Solution B4) Sample solution P Papain PBS solution: 300 U/mL Tween20 PBS solution: 10% by weight

(Sample Solution B5) Sample solution P Papain PBS solution: 300 U/mL WST-3 water solution: 2% by weight

(Sample Solution B6) Sample solution P Papain PBS solution: 300 U/mL WST-4 PBS solution: 0.5% by weight

(Sample Solution B7) Sample solution P Papain PBS solution: 300 U/mL WST-5 PBS solution: 2% by weight

(Sample Solution B8) Sample solution P Papain PBS solution: 300 U/mL TTAB 10%: by weight WST-3 PBS solution: 2% by weight

(Sample Solution B9) Sample solution P Papain PBS solution: 300 U/mL TTAB 10%: by weight WST-4 PBS solution: 0.5% by weight

(Sample Solution B10) Sample solution P Papain PBS solution: 300 U/mL TTAB: 10% by weight WST-5 PBS solution: 2% by weight

(Sample Solution B11) Sample solution P Papain PBS solution: 300 U/mL TritonX-100 PBS solution: 10% by weight WST-3 PBS solution: 2% by weight

(Sample Solution B12) Sample solution P Papain PBS solution: 300 U/mL TritonX-100 PBS solution: 10% by weight WST-4 PBS solution: 0.5% by weight

(Sample Solution B13) Sample solution P Papain PBS solution: 300 U/mL TritonX-100 PBS solution: 10% by weight WST-5 PBS solution: 2% by weight

(Sample Solution B14) Sample solution P Papain PBS solution: 300 U/mL Tween20 PBS solution: 10% by weight WST-3 PBS solution: 2% by weight

(Sample Solution B15) Sample solution P Papain PBS solution: 300 U/mL Tween20 PBS solution: 10% by weight WST-4 PBS solution 0.5%: by weight

(Sample Solution B16)

Sample solution P Papain PBS solution: 300 U/mL Tween20 PBS solution: 10% by weight WST-5 PBS solution: 2% by weight

(Comparative Sample Solution b1) Sample solution P Papain PBS solution: 300 U/mL

(HbA1c Decompsition Reaction Using Papain)

The temperature of the sample solutions B1 - B16 and the comparative sample solution b1 was maintained at 37 degrees Celsius for ten minutes and the reaction occurred.

(Comparison of the Decomposition Reaction Progress)

After the reaction, the sample solutions B1-B16 and the comparative sample solution b1 were subjected to polyacrylamide gel electrophoresis (PAGE). FIG. 2A and FIG. 2B show the electrophoresis patterns. The character “M” depicted in FIG. 2A and FIG. 2B indicates a protein maker.

The degree of the HbA1c decomposition is reflected in the color strength of the band near 64.5 KDa, which corresponds to hemoglobin molecular region included in the electrophoresis pattern. If the color strength of the band near 64.5 KDa decreases, the more amounts of HbA1c is decomposed.

As is clear from the electrophoresis patterns shown in FIG. 2A and FIG. 2B, the more amount of HbA1c was decomposed in the sample solutions B1, B8, B9 and B10, compared to the comparative sample solution b1. The more amount of HbA1c was decomposed in the sample solutions B8, B9 and B10, compared to the sample solutions B2, B5, B6, and B7.

SDS (Sample solution B1), the composition of TTAB and WST-3 (Sample solution B8), the composition of TTAB and WST-4 (Sample solution B9) and the composition of TTAB and WST-5 (Sample solution B10) improve the decomposition rate of HbA1c using papain.

The composition of TTAB and WST-3 (Sample solution B8) improves the decomposition rate of HbA1c using papain, compared to the case where TTAB was used alone (Sample solution B2) and the case where WST-3 was used alone (Sample solution B5).

The composition of TTAB and WST-4 (Sample solution B9) improves the decomposition rate of HbA1c using papain, compared to the case where TTAB was used alone (Sample solution B2) and the case where WST-4 was used alone (Sample solution B6).

The composition of TTAB and WST-5 (Sample solution B10) improves the decomposition rate of HbA1c using papain, compared to the case where TTAB was used alone (Sample solution B2) and the case where WST-5 was used alone (Sample solution B7).

(Test Example 3) (Preparation of the Sample Solution)

The following sample solutions C1-C12 were prepared. A comparative sample solution c1 was also prepared. The source of the used reagents is listed below.

FPOX-CE: Kikkoman Corporation SDS: Wako Pure Chemical Industries, Ltd. Peroxidase: Wako Pure Chemical Industries, Ltd. KN-111 (color forming dye): Dojindo Laboratories TTAB (tetradecyl trimethyl ammonium bromide): Wako Pure Chemical Industries, Ltd. TritonX-100: Wako Pure Chemical Industries, Ltd. Tween20: Wako Pure Chemical Industries, Ltd. WST-3: Dojindo Laboratories WST-4: Dojindo Laboratories WST-5: Dojindo Laboratories

(Sample Solution C1) SDS PBS solution: 10% by weight FPOX-CE PBS solution: 28 U/mL Peroxidase 20 U/mL KN-111: 2 mM

(Sample Solution C2) TTAB: 10% by weight FPOX-CE PBS solution: 28 U/mL Peroxidase 20 U/mL KN-111: 2 mM

(Sample Solution C3) TritonX-100 PBS solution: 10% by weight FPOX-CE PBS solution: 28 U/mL Peroxidase: 20 U/mL KN-111: 2 mM

(Sample Solution C4) Tween20 PBS solution: 10% by weight FPOX-CE PBS solution: 28 U/mL Peroxidase: 20 U/mL KN-111: 2 mM

(Sample Solution C5) WST-3 PBS solution: 2% by weight FPOX-CE PBS solution: 28 U/mL Peroxidase: 20 U/mL KN-111: 2 mM

(Sample Solution C6) WST-4 PBS solution: 0.5% by weight FPOX-CE PBS solution: 28 U/mL Peroxidase 20 U/mL KN-111: 2 mM

(Sample Solution C7) WST-5 PBS solution: 2% by weight FPOX-CE PBS solution: 28 U/mL Peroxidase: 20 U/mL KN-111: 2 mM

(Sample Solution C8) TTAB: 10% by weight WST-3 PBS solution: 2% by weight FPOX-CE PBS solution: 28 U/mL Peroxidase: 20 U/mL KN-111: 2 mM

(Sample Solution C9) TTAB: 10% by weight WST-4 PBS solution: 0.5% by weigh FPOX-CE PBS solution: 28 U/mL Peroxidase: 20 U/mL KN-111: 2 mM

(Sample Solution C10) TTAB: 10% by weight WST-5 PBS solution: 2% by weigh FPOX-CE PBS solution: 28 U/mL Peroxidase: 20 U/mL KN-111: 2 mM

(Sample Solution C11) TritonX-100 PBS solution: 10% by weight WST-3 PBS solution: 2% by weight FPOX-CE PBS solution: 28 U/mL Peroxidase: 20 U/mL KN-111: 2 mM

(Sample Solution C12) Tween20 PBS solution: 10% by weight WST-3 PBS solution: 2% by weight FPOX-CE PBS solution: 28 U/mL Peroxidase: 20 U/mL KN-111: 2 mM

(Comparative Sample Solution c1) FPOX-CE PBS solution: 28 U/mL Peroxidase: 20 U/mL KN-111: 2 mM

(Reaction of the Glycated Peptide with FPOX)

The temperature of the sample solutions C1-C12 and the comparative sample solution c1 was maintained at 37 degrees Celsius for ten minutes and the reaction occurred.

After the reaction, five milliliter of Fru-Val-His 1 mM PBS solution (Available from Peptide Institute Inc.) was each added to the sample solution C1-C12 and comparative sample solution c1 (95 microliter). In this way, mixture solutions C1-C12 and a comparative mixture solution c1 were obtained.

(Evaluation of FPOX Activity)

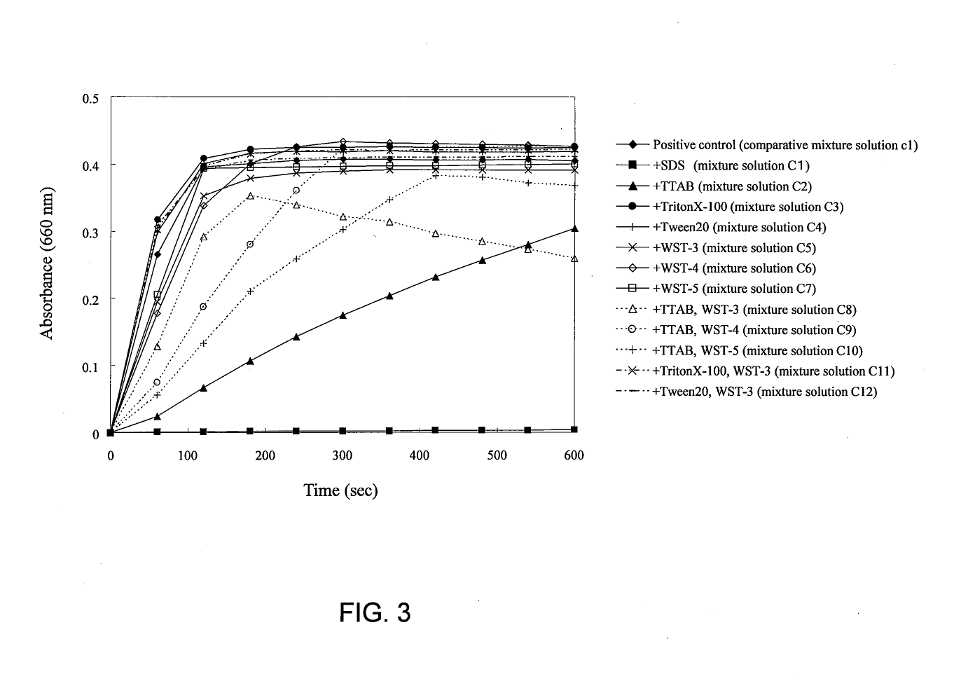

Absorbance of the mixture solutions C1-C12 and the comparative mixture solution c1 at a wavelength of 660 nanometers was measured every minute with an absorption spectrometer (available from Tecan Group Inc., under the trade name of Infinite 200 Pro). FIG. 3 shows the results.

The glycated peptide Fru-Val-His reacts with the FPOX to generate hydrogen peroxide. Hydrogen peroxide reacts with the color forming dye KN-111 to change the color forming dye KN-111 into red. Accordingly, the activity of the FPOX is evaluated by measuring the absorbance of the color forming dye KN-111 at a wavelength of 660 nanometers (red).

As shown in FIG. 3, an absorbance change was observed in all of the mixture solutions C2-C12 other than the mixture solution C1 and the comparative sample solution c1. Among them, it was observed that the absorbance changes were similar to that of the comparative mixture solution c1, other than the mixture solution C2. It was observed that the absorbance change of the mixture solution C2 was more gradual than that of the comparative mixture solution c1.

On the other hand, in the sample solution C1, the absorbance change was not observed at all.

In this way, SDS, which is an anionic surfactant, inactivates FPOX (see the result of the mixture solution C1). Therefore, SDS is not allowed to be used for the present quantification method of HbA1c using the FPOX. On the other hand, a cationic surfactant and a non-ionic surfactant do not inactivate FPOX (see the results of the mixture solutions C2-C12 and the comparative mixture solution c1).

As is clear from FIG. 3, the FPOX reacts more rapidly with the glycated peptide in the case where the composition of TTAB and WST-3, the composition of TTAB and WST-4, or the composition of TTAB and WST-5 is used (mixture solutions C8-C10), compared to the case where TTAB is used alone (mixture solution C2).

(Test Example 4)

(Preparation of the Sample Solution)

The following sample solutions D1-D12 were prepared. A comparative sample solution d1 was also prepared. The source of the used reagents is listed below. FPOX-CE: Kikkoman Corporation Thermolysin: Wako Pure Chemical Industries, Ltd. SDS: Wako Pure Chemical Industries, Ltd. Peroxidase Wako Pure Chemical Industries, Ltd. KN-111 (color forming dye): Dojindo Laboratories TTAB (tetradecyl trimethyl ammonium bromide): Wako Pure Chemical Industries, Ltd. TritonX-100: Wako Pure Chemical Industries, Ltd. Tween20: Wako Pure Chemical Industries, Ltd. WST-3: Dojindo Laboratories WST-4: Dojindo Laboratories WST-5: Dojindo Laboratories

(Sample Solution D1) Thermolysin PBS solution: 150,000 U/mL SDS PBS solution: 10% by weight FPOX-CE PBS solution: 28 U/mL Peroxidase: 20 U/mL KN-111: 2 mM

(Sample Solution D2) Thermolysin PBS solution: 150,000 U/mL TTAB: 10% by weight FPOX-CE PBS solution: 28 U/mL Peroxidase: 20 U/mL KN-111: 2 mM

(Sample Solution D3) Thermolysin PBS solution: 150,000 U/mL TritonX-100 PBS solution: 10% by weight FPOX-CE PBS solution: 28 U/mL Peroxidase: 20 U/mL KN-111: 2 mM

(Sample Solution D4) Thermolysin PBS solution: 150,000 U/mL Tween20 PBS solution: 10% by weight FPOX-CE PBS solution: 28 U/mL Peroxidase: 20 U/mL KN-111: 2 mM

(Sample Solution D5) Thermolysin PBS solution: 150,000 U/mL WST-3 PBS solution: 2% by weight FPOX-CE PBS solution: 28 U/mL Peroxidase: 20 U/mL KN-111: 2 mM

(Sample Solution D6) Thermolysin PBS solution: 150,000 U/mL WST-4 PBS solution: 0.5% by weight FPOX-CE PBS solution: 28 U/mL Peroxidase: 20 U/mL KN-111: 2 mM

(Sample Solution D7) Thermolysin PBS solution: 150,000 U/mL WST-5 PBS solution: 2% by weight FPOX-CE PBS solution: 28 U/mL Peroxidase: 20 U/mL KN-111: 2 mM

(Sample Solution D8) Thermolysin PBS solution: 150,000 U/mL TTAB: 10% by weight WST-3 PBS solution: 2% by weight FPOX-CE PBS solution: 28 U/mL Peroxidase: 20 U/mL KN-111: 2 mM

(Sample Solution D9) Thermolysin PBS solution: 150,000 U/mL TTAB: 10% by weight WST-4 PBS solution: 0.5% by weigh FPOX-CE PBS solution: 28 U/mL Peroxidase: 20 U/mL KN-111: 2 mM

(Sample Solution D10) Thermolysin PBS solution: 150,000 U/mL TTAB: 10% by weight WST-5 PBS solution: 2% by weigh FPOX-CE PBS solution: 28 U/mL Peroxidase: 20 U/mL KN-111: 2 mM

(Sample Solution D11) Thermolysin PBS solution: 150,000 U/mL TritonX-100 PBS solution: 10% by weight WST-3 PBS solution: 2% by weight FPOX-CE PBS solution: 28 U/mL Peroxidase: 20 U/mL KN-111: 2 mM

(Sample Solution D12) Thermolysin PBS solution: 150,000 U/mL Tween20 PBS solution: 10% by weight WST-3 PBS solution: 2% by weight FPOX-CE PBS solution: 28 U/mL Peroxidase: 20 U/mL KN-111: 2 mM

(Comparative Sample Solution c1) Thermolysin PBS solution: 150,000 U/mL FPOX-CE PBS solution: 28 U/mL Peroxidase: 20 U/mL KN-111: 2 mM

(Reaction of the Glycated Peptide with FPOX)

The temperature of the sample solutions D1-D12 and the comparative sample solution d1 was maintained at 37 degrees Celsius for ten minutes and the reaction occurred.

After the reaction, five milliliter of Fru-Val-His 1 mM PBS solution (Available from Peptide Institute Inc.) was each added to the sample solution D1-D12 and comparative sample solution d1 (95 microliters). In this way, mixture solutions D1-D12 and a comparative mixture solution dl were obtained.

(Evaluation of FPOX Activity)

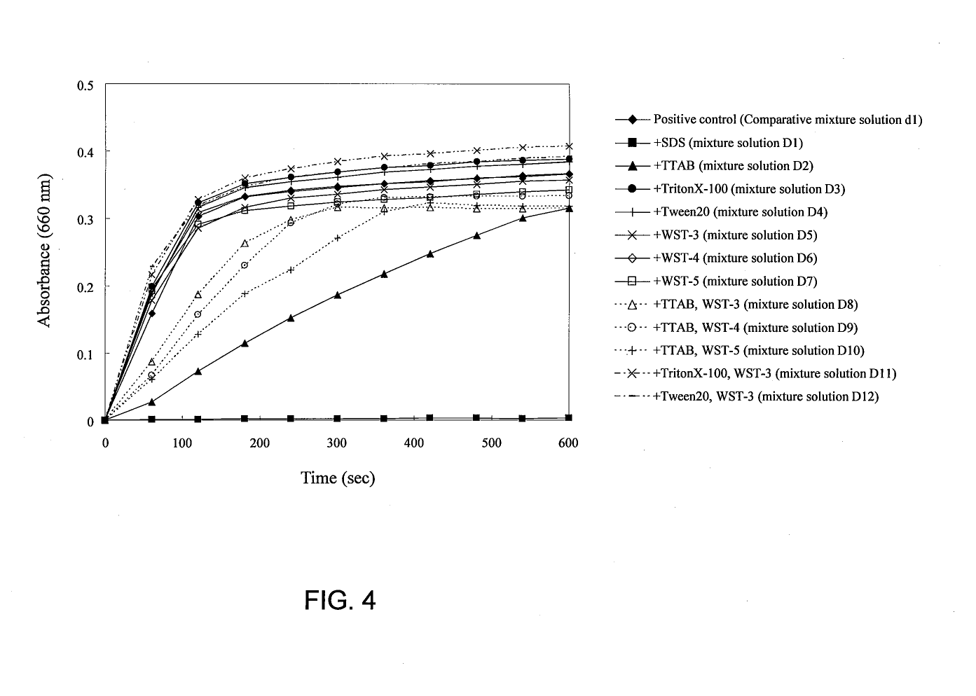

Absorbance of the mixture solutions D1-D12 and the comparative mixture solution d1 at a wavelength of 660 nanometers was measured every minute with an absorption spectrometer (available from Tecan Group Inc., under the trade name of Infinite 200 Pro). FIG. 4 shows the results.

The glycated peptide Fru-Val-His reacts with the FPOX to generate hydrogen peroxide. Hydrogen peroxide reacts with the color forming dye KN-111 to change the color forming dye KN-111 into red. Accordingly, the activity of the FPOX is evaluated by measuring the absorbance of the color forming dye KN-111 at a wavelength of 660 nanometers (red).

As shown in FIG. 4, an absorbance change was observed in all of the mixture solutions D2-D12 other than the mixture solution D1 and the comparative sample solution d1. Among them, it was observed that the absorbance changes were similar to that of the comparative mixture solution d1, other than the mixture solution D2. It was observed that the absorbance change of the mixture solution D2 was more gradual than that of the comparative mixture solution d1.

On the other hand, in the sample solution D1, the absorbance change was not observed at all.

In this way, SDS, which is an anionic surfactant, inactivates FPOX (see the result of the mixture solution D1). Therefore, SDS is not allowed to be used for a quantification method of HbA1c using the FPOX. On the other hand, a cationic surfactant and a non-ionic surfactant do not inactivate FPOX (see the results of the mixture solution D2-D12 and the comparative mixture solution c1). Thermolysin also does not inactivate the FPOX.

As is clear from FIG. 4, the FPOX reacts more rapidly with the glycated peptide in the case where the composition of TTAB and WST-3, the composition of TTAB and WST-4, or the composition of TTAB and WST-5 is used (mixture solutions D8-D10), compared to the case where TTAB is used alone (mixture solution D2).

(Test Example 5)

(Preparation of the Sample Solution)

The following sample solutions E1-E12 were prepared. A comparative sample solution e1 was also prepared. The source of the used reagents is listed below.

FPOX-CE: Kikkoman Corporation Papain: Roche Diagnostics K.K. SDS: Wako Pure Chemical Industries, Ltd. Peroxidase: Wako Pure Chemical Industries, Ltd. KN-111 (color forming dye): Dojindo Laboratories TTAB (tetradecyl trimethyl ammonium bromide): Wako Pure Chemical Industries, Ltd. TritonX-100: Wako Pure Chemical Industries, Ltd. Tween20: Wako Pure Chemical Industries, Ltd. WST-3: Dojindo Laboratories WST-4: Dojindo Laboratories WST-5: Dojindo Laboratories

(Sample Solution E1) Papain 300 U/mL SDS PBS solution: 10% by weight FPOX-CE PBS solution: 28 U/mL Peroxidase 20 U/mL KN-111: 2 mM

(Sample Solution E2) Papain: 300 U/mL TTAB: 10% by weight FPOX-CE PBS solution: 28 U/mL Peroxidase: 20 U/mL KN-111: 2 mM

(Sample Solution E3) Papain: 300 U/mL TritonX-100 PBS solution: 10% by weight FPOX-CE PBS solution: 28 U/mL Peroxidase: 20 U/mL KN-111: 2 mM

(Sample Solution E4) Papain: 300 U/mL Tween20 PBS solution: 10% by weight FPOX-CE PBS solution: 28 U/mL Peroxidase: 20 U/mL KN-111: 2 mM

(Sample Solution E5) Papain: 300 U/mL WST-3 PBS solution: 2% by weight FPOX-CE PBS solution: 28 U/mL Peroxidase: 20 U/mL KN-111: 2 mM

(Sample Solution E6) Papain: 300 U/mL WST-4 PBS solution: 0.5% by weight FPOX-CE PBS solution: 28 U/mL Peroxidase: 20 U/mL KN-111: 2 mM

(Sample Solution E7) Papain: 300 U/mL WST-5 PBS solution: 2% by weight FPOX-CE PBS solution: 28 U/mL Peroxidase: 20 U/mL KN-111: 2 mM

(Sample Solution E8) Papain: 300 U/mL TTAB: 10% by weight WST-3 PBS solution: 2% by weight FPOX-CE PBS solution: 28 U/mL Peroxidase: 20 U/mL KN-111: 2 mM

(Sample Solution E9) Papain: 300 U/mL TTAB: 10% by weight WST-4 PBS solution: 0.5% by weigh FPOX-CE PBS solution: 28 U/mL Peroxidase: 20 U/mL KN-111: 2 mM

(Sample Solution E10) Papain: 300 U/mL TTAB: 10% by weight WST-5 PBS solution: 2% by weigh FPOX-CE PBS solution: 28 U/mL Peroxidase: 20 U/mL KN-111: 2 mM

(Sample Solution E11) Papain: 300 U/mL TritonX-100 PBS solution: 10% by weight WST-3 PBS solution: 2% by weight FPOX-CE PBS solution: 28 U/mL Peroxidase: 20 U/mL KN-111: 2 mM

(Sample Solution E12) Papain: 300 U/mL Tween20 PBS solution: 10% by weight WST-3 PBS solution: 2% by weight FPOX-CE PBS solution: 28 U/mL Peroxidase 20 U/mL KN-111: 2 mM

(Comparative Sample Solution c1) Thermolysin PBS solution: 150,000 U/mL FPOX-CE PBS solution: 28 U/mL Peroxidase: 20 U/mL KN-111: 2 mM

(Reaction of the Glycated Peptide with FPOX)

The temperature of the sample solutions E1-E12 and the comparative sample solution el was maintained at 37 degrees Celsius for ten minutes and the reaction occurred.

After the reaction, five milliliter of Fru-Val-His 1 mM PBS solution (Available from Peptide Institute Inc.) was each added to the sample solution E1-E12 and comparative sample solution e1 (95 microliters). In this way, mixture solutions E1-E12 and a comparative mixture solution e1 were obtained.

(Evaluation of FPOX Activity)

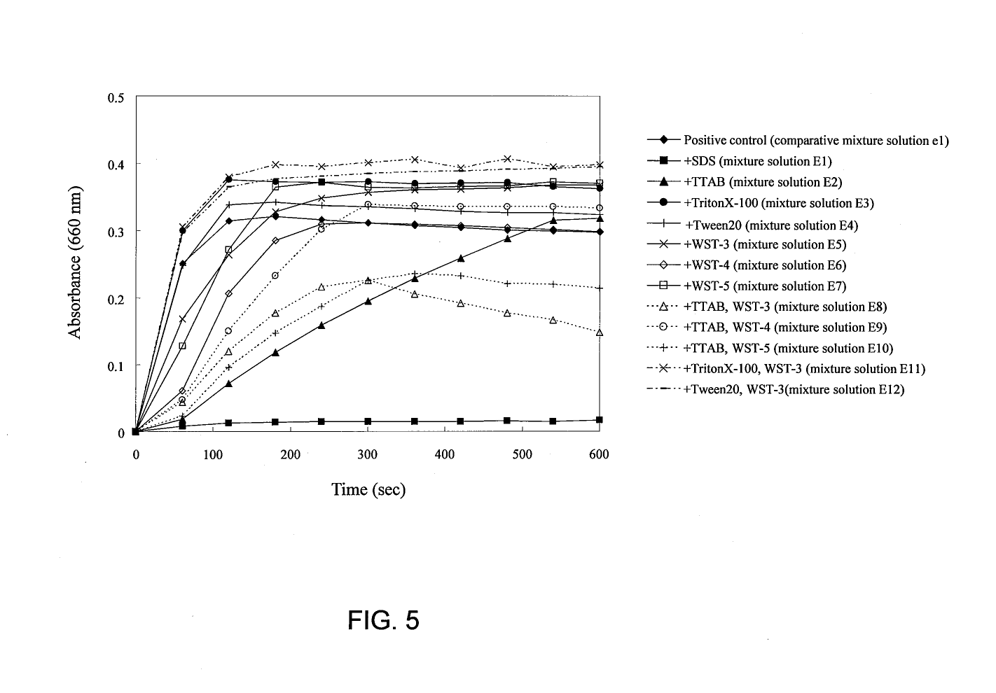

Absorbance of the mixture solutions E1-E12 and the comparative mixture solution e1 at a wavelength of 660 nanometers was measured every minute with an absorption spectrometer (available from Tecan Group Inc., under the trade name of Infinite 200 Pro). FIG. 5 shows the results.

The glycated peptide Fru-Val-His reacts with the FPOX to generate hydrogen peroxide. Hydrogen peroxide reacts with the color forming dye KN-111 to change the color forming dye KN-111 into red. Accordingly, the activity of the FPOX is evaluated by measuring the absorbance of the color forming dye KN-111 at a wavelength of 660 nanometers (red).

As shown in FIG. 5, an absorbance change was observed in all of the mixture solutions E2-E12 other than the mixture solution E1 and the comparative sample solution e1. Among them, it was observed that the absorbance changes were similar to that of the comparative mixture solution e1, other than the mixture solution E2. It was observed that the absorbance change of the mixture solution E2 was more gradual than that of the comparative mixture solution e1.

On the other hand, in the sample solution E1, the absorbance change was not observed at all.

In this way, SDS, which is an anionic surfactant, inactivates FPOX (see the result of the mixture solution E1). Therefore, SDS is not allowed to be used for a quantification method of HbA1c using the FPOX. On the other hand, a cationic surfactant and a non-ionic surfactant do not inactivate FPOX (see the results of the mixture solution E2-E12 and the comparative mixture solution c1). Papain also does not inactivate the FPOX.

As is clear from FIG. 5, the FPOX reacts more rapidly with the glycated peptide in the case where the composition of TTAB and WST-3, the composition of TTAB and WST-4, or the composition of TTAB and WST-5 is used (mixture solutions E8-E10), compared to the case where TTAB is used alone (mixture solution E2).

### INDUSTRIAL APPLICABILITY

The present invention is useful for diagnosis of diabetes.

1-14. (canceled)

15. A method for quantifying glycated hemoglobin (HbA1c) contained in a sample, the method comprising steps of:
(a) mixing the sample with a protease in the presence of a composition of a cationic surfactant and a tetrazolium salt to obtain a glycated-peptide aqueous solution containing a glycated peptide;
(b) mixing the glycated-peptide aqueous solution obtained in the step (a) with fluctosyl peptide oxidase to obtain hydrogen peroxide; wherein
the glycated-peptide aqueous solution contains the composition; and
(c) calculating a concentration of the glycated hemoglobin (HbA1c) on the basis of an amount of the hydrogen peroxide obtained in the step (b), wherein the cationic surfactant is a quaternary ammonium salt, which is represented by the chemical formula R₁R₂R₃R₄N⁺X⁻: wherein R₁ is an alkyl group having a carbon number of not less than 8 and not more than 18; R₂, R₃ and R₄ are lower alkyl groups independently; and X represents halogens.

16. The method according to claim 15, wherein
the protease is selected from the group consisting of thermolysin and papain.

17. The method according to claim 15, wherein
the tetrazolium salt is selected from the group consisting of 2-(4-Iodophenyl)-3-(2,4-dinitrophenyl)-5-(2,4-disulfophenyl)-2H-tetrazolium, monosodium salt,
2-(2-Benzothiazolyl)-3-(4-carboxy-2-methoxyphenyl)-5-[4-[(2-sodiosulfoethyl)carbamoyl]phenyl]-2H-tetrazol-3-ium, and
2,2′-(3,3′-dimethoxy-4,4′-biphenylylene) bis[3-(2-benzothiazolyl) -5[4-[N-[2-(sodiooxysulfonyl) ethyl]-N-(2- sulfoethyl) carbamoyl]phenyl]-2H-tetrazole-3-ium].

18. The method according to claim 15, wherein
the glycated peptide is a fluctosyl peptide.

19. The method according to claim 18, wherein
the fluctosyl peptide is fluctosyl-valine-histidine.

20. A method for quantifying glycated hemoglobin (HbA1c) contained in a sample, the method comprising steps of:
(a) mixing the sample with a protease and fluctosyl peptide oxidase in the presence of a composition of a cationic surfactant and a tetrazolium salt to obtain hydrogen peroxide; and
(b) calculating a concentration of the glycated hemoglobin (HbA1c) on the basis of an amount of the hydrogen peroxide obtained in the step (a), wherein the cationic surfactant is a quaternary ammonium salt, which is represented by the chemical formula R₁R₂R₃R₄N⁺X⁻: wherein R₁ is an alkyl group having a carbon number of not less than 8 and not more than 18; R₂, R₃ and R₄ are lower alkyl groups independently; and X represents halogens.

21. The method according to claim 20, wherein
the protease is selected from the group consisting of thermolysin and papain.

22. The method according to claim 20, wherein
the tetrazolium salt is selected from the group consisting of
2-(4-Iodophenyl)-3-(2,4-dinitrophenyl)-5-(2,4-disulfophenyl)- 2H-tetrazolium, monosodium salt,
2-(2-Benzothiazolyl)-3-(4-carboxy-2-methoxyphenyl)-5-[4-[(2-sodiosulfoethyl) carbamoyl]phenyl]-2H-tetrazol-3-ium, and
2,2′-(3,3′-dimethoxy-4,4′-biphenylylene) bis[3-(2-benzothiazolyl)-5[4-[N-[2-(sodiooxysulfonyl) ethyl]-N-(2-sulfoethyl) carbamoyl]phenyl]-2H- tetrazole-3-ium].

23. The method according to claim 20, wherein
the glycated peptide is a fluctosyl peptide.

24. The method according to claim 23, wherein
the fluctosyl peptide is fluctosyl-valine-histidine.

25. A composition for activating protease, containing
a cationic surfactant and
a tetrazolium salt, wherein the cationic surfactant is a quaternary ammonium salt, which is represented by the chemical formula R₁R₂R₃R₄N⁺X⁻: wherein R₁ is an alkyl group having a carbon number of not less than 8 and not more than 18; R₂, R₃ and R₄ are lower alkyl groups independently; and X represents halogens.

26. The composition according to claim 25, wherein
the protease is selected from the group consisting of thermolysin and papain.
